(19) United States
(12) Patent Application Publication
(10) Pub. No.: US 2022/0218817 A1
(43) Pub. Date: Jul. 14, 2022

(54) IMMUNE MODULATION BY MESENCHYMAL STEM CELLS

(71) Applicant: Vitro Biopharma, Inc., Golden, CO (US)

(72) Inventors: Jack Zamora, Denver, CO (US); Tiana States, Wheatridge, CO (US); James Musick, Golden, CO (US)

(21) Appl. No.: 17/647,401

(22) Filed: Jan. 7, 2022

Related U.S. Application Data
(60) Provisional application No. 63/135,441, filed on Jan. 8, 2021.

(51) Int. Cl. A61K 39/215; A61P 31/14; A61K 35/28; C12N 5/0775; A61K 39/39; C12N 7/00

(52) U.S. Cl. CPC A61K 39/215; A61P 31/14; C12N 7/00; C12N 5/0665; A61K 39/39; A61K 35/28

(57) ABSTRACT

A method is disclosed of treating a patient having viral pneumonia secondary to COVID-19 using IV infusion of mesenchymal stem cells in patients receiving COVID-19 vaccination. The vaccine may be Pfizer, Moderna, Johnson & Johnson or Astra Zenica COVID-19 vaccine, for example and the patient may be treated with an mRNA-based vaccine followed by IV-infusion of 100 million MSCs. MSCs can be derived from patients who have been exposed to the COVID-19 virus. Moreover, the MSCs may be derived from the umbilical cord of a donor.

### CROSS REFERENCE TO RELATED APPLICATIONS

This application claims priority to U.S. Provisional Application No. 63/135,441 filed Jan. 8, 2021, the entire contents of which are incorporated herein by reference.

### FIELD OF THE INVENTION

The present invention is related to the use of mesenchymal stem cells (MSCs) and, more particularly, to modulation of immune responses including prevention, treatment and control of viral infection by MSCs and various MSC formulations and adjunctive therapies that specifically modulate immune responses in a patient in need.

### BACKGROUND OF THE INVENTION

Mesenchymal stem cells (MSCs) are particularly relevant to the present invention. These cells were initially described by Arnold Caplan in 2001. MSCs may be derived from various tissues including bone marrow, adipose-tissue, dental pulp, umbilical cord, amniotic fluid, membranes, placenta, and other sources well-known to those skilled in the art. Comparative studies suggest differences in potency, differentiation capacity, growth rates, and other cellular characteristics depending on the tissue source used to procure MSCs. Such characteristics may be related to several factors, including medical status and age of the donor as well as environmental factors specific to the stem cell niche together with cell culture conditions used during the manufacturing process.

MSCs derived from bone marrow and adipose tissue have been subject to numerous clinical trials that provide preliminary evidence of safety and efficacy. A classic feature of MSCs is trilineage differentiation into adipocytes, chondrocytes, and osteoblasts, although several other cellular lineages may be derived from MSCs, including neural, kidney, and cardiac cells. Hence, MSCs have been extensively studied in musculoskeletal conditions such as osteoarthritis (OA). Several studies support safety and efficacy by intra-articular injections into knees, hips, and shoulder joints of OA patients. In addition, several other conditions may be treated by MSC transplants or transplants of progenitor cells derived from MSCs including stroke, myocardial infarct, congestive heart failure, along with several other indications.

Severe acute respiratory syndrome coronavirus 2 (SARS-CoV-2), the cause of coronavirus pandemic can result in a severe respiratory illness requiring prolonged ventilatory support. For these critically ill patients, the case fatality rate can be as high as 37% with variations in different sample populations, specific COVID-19 variants and other factors. Some severe Covid-19 pneumonia patients are treated with venovenous extracorporeal membrane oxygenation (ECMO) with similar survival rates. Damage to the pulmonary tissue is caused by both SARS-CoV-2 as well as the immune response to the virus. Anti-inflammatory treatment, such as the use of the steroid dexamethasone, results in reduced mortality for critically ill patients. (Horby, P, et al, Dexamethasone in Hospitalized Patients with COVID-19. N Eng. J. Med 2021; 384:693-704)

MSCs have the ability to migrate to regions of active inflammation and exert and anti-inflammatory effect through chemokine ligand/receptor interactions. Furthermore, MSCs can differentiate into various lung cells to help replace cells damaged by SARS-CoV-2 or the subsequent inflammation and regenerate lung tissue as well.

There is evidence that MSCs have clinical utility in the treatment of COVID-19 infections. Stem cells resist viral attack by the expression of interferon gamma stimulated genes (ISGs). These are expressed in stem cells prior to their differentiation. Hence, stem cells would be expected to survive even if transplanted into a patient with an active coronavirus infection. Also, it is known that stem cells regenerate cells through various processes involving reduction of inflammation, secretion of substances that protect cells, transfer of mitochondria, anti-apoptosis/anti-oxidative effects and modulation of the immune system.

Stem cells migrate to sites of inflammation through a system of chemokine signaling ligands and receptors including the SDF1a/CXCR4 axis. An initial shift from pro-inflammatory to anti-inflammatory cytokines occurs, through inhibition of IL-1beta. Stem cells also shift the M1 macrophage phenotype to M2. The M1 phenotype is pro-inflammatory and the M2 phenotype is anti-inflammatory. Stem cells thus provide powerful anti-inflammatory effects by both molecular and cellular effects. MSCs modulate immune function by immunosuppression, induction of TREG cells, suppression of cytokine-activated T cells, and activation of dendritic cells. Depending on local factors in particular niches, MSC can also activate immune responses. (Fan, X L, et al, Cellular and Molecular Life Sciences (2020) 77:2771-2794)

These properties of MSCs are likely to underly therapeutic benefits in treating COVID-19 infections by blocking the cytokine cascade, regenerating cells and reversing multi-organ failure. These effects have been shown to increase survival rates in patients who have been infected with coronavirus.

Pre-clinical evidence also indicates stem cell protection against viral infection. Influenza virus A/H5N1 causes acute lung injury that was reduced by human MSCs in mice and the treatment increased survival (Yudhawati, et al, BMC Infect Dis 2020 Nov. 11; 20(1):823). The primary mechanism of therapeutic benefit is not necessarily clearance of viral load in COVID-19 patients but rather anti-inflammatory and regenerative effects that correct organ and tissue damage caused by the COVID-19 virus and other viruses that attack the lungs and other organs.

As opposed to vaccines, stem cell therapy provides alternative therapeutic benefits in cellular/organ system regeneration without being specific merely to COVID-19 or any of its variant sequences caused by natural mutations. Rather, stem cell therapy is relevant to a broad range of viral infections that induce acute respiratory distress (ARDS), including SARS-CoV, SARS-CoV-2 (COVID-19), MERS-CoV, influenza viruses including H5N1, herpes simplex virus, cytomegalovirus (CMV) and other known and unknown viruses that induce ARDS.

While MSC therapy does have anti-viral effects (Khatri, M. et al, Stem Cell Res Ther 9:17, 2018), this is not necessarily the sole mechanism of viral clearance since MSC therapy includes various mechanisms of immunomodulation that may eliminate viruses. In addition, stem cells are known to be resistant to viral attack by expression of interferon gamma stimulated genes (ISGs), which are expressed in stem cells prior to their differentiation (Wu, X. et al. Cell 172, 423-438.e25 2018; Khoury, M. et al. Eur Respir J 55, 2000858 (2020). Included in this set of genes are IF16, ISG15, SAT1, PMAIP1, p21/CDKN1A and CCL2, as well as the IFITM family proteins, members of which are unique in that they prevent infection before a virus can traverse the lipid bilayer of the cell. In addition, stem cell immunity to RNA viruses is further enhanced by a newly discovered isoform of Dicer that initiates potent antiviral RNA interference (Poirier, E. Z. et al. Science 373, 231-236 (2021).

The detailed mechanism of action of MSC therapy in COVID-19 infections appears to involve multiple clinical benefits including improved pulmonary, renal, and hepatic function, anti-coagulation effects and viral clearance. (Li, Z, et al, Cell Prolif 53: e12939, 2020; Al-Khawega, S F and Abdelalim, E M, Stem Cell Res Ther. 11:437, 2020). As such, it is likely that MSC therapy is effective in treating prolonged symptoms following COVID-19 infection known as PASC (post-acute sequelae SARS-CoV-2 infection) or long COVID. The results shown in example 1 supports this hypothesis by showing recovery from renal, hepatic and pulmonary failure following MSC IV infusion. A recent study supports an autoimmune pathophysiology underlying PASC, long COVID-19 (Liu, et al, J. Transl. Med., (2021) 19: 524) and given the immunosuppressive mechanism of active of MSC therapy including PGE2 secretion that inhibit T cells (Fan, X L, et al, Cellular and Molecular Life Sciences (2020) 77:2771-2794), MSC therapy of PASC, long COVID-19 is further supported.

There have been 6 separate clinical reports so far with a total of 115 patients treated by IV infusion of MSCs—providing evidence of safety and efficacy of MSC therapy for COVID-19 in humans: An initial study of 7 patients treated by IV infusion of 10⁶ MSCs/Kg body weight umbilical cord-derived MSCs showed resolution of symptoms including: improved pulmonary function, increased peripheral lymphocytes and decreased cytokine-activated T & NK cells, increased CD14+, CD11c+, CD11b^{mid+} dendritic cells, and a shift from pro- to anti-inflammatory cytokines (Leng, Z. et al. Transplantation of ACE2- Mesenchymal Stem Cells Improves the Outcome of Patients with COVID-19 Pneumonia. Aging Dis 11, 216-228 (2020). An Israeli biotech firm “Pluristem”, has also reported positive results in the treatment of 6 infected patients with COVID-19 who were in acute respiratory distress. The therapy called PLX Cell Therapy consists of allogeneic MSC-like cells with immunomodulatory properties including increased M2 macrophages. After one week of treatment, all 6 patients survived and four 4 showed improved respiratory functions. (Papait, A. et al. Front Bioeng Biotechnol 8, 619980 (2021).

The Australian firm “Mesoblast” reported positive outcomes in 12 infected patients with COVID-19 who were treated with allogeneic MSCs through compassionate use in Mt. Sinai Hospital in NYC. The results showed 83% survival after two IV infusions of Remestemcel-L while a comparable control group showed 9% survival using the standard of care. (Gelijns, A. Mesenchymal Stromal Cells for the Treatment of Moderate to Severe COVID-19 Acute Respiratory Distress Syndrome. https://clinicaltrials.gov/ct2/show/NCT04371393 (2021).

A study on the treatment of 13 COVID-19 patients with adipose-derived MSCs at 1 million MSCs/Kg body weight reported no adverse events and clinical improvements in 70% of the patients including reduction in CRP, IL-6, ferritin, LDH & D-dimer together with increased lymphocytes. (Sánchez-Guijo, F. et al. EClinicalMedicine 25, (2020).

A randomized, placebo-controlled trial conducted by the Miller School of Medicine included 24 patients randomized to treatment by umbilical cord MSCs or placebo. No significant adverse events and a 91% survival in the treated group was observed, with only 42% (p=0.015) survival in the control (Lanzoni, G. et al. STEM CELLS Translational Medicine 10, 660-673 (2021).

Another randomized, double-blind, placebo-controlled phase 2 trial in 65 UC-MSC treated patients verses 35 placebo patients showed significant improvements in pulmonary function based on altered portions of whole lung lesion volumes, reduction in solid component of lesion volume and increased six-minute walk test without alteration in adverse events (Shi, L. et al. Sig Transduct Target Ther 6, 1-9 (2021).

### SUMMARY OF THE INVENTION

The prior art shows safety and efficacy in the treatment of COVID-19 infections by IV infusions of MSCs. The present invention teaches new methods to enhance the therapeutic benefit of MSCs by the formulation of MSCs with enhanced immune responses to viral infections by conferring vaccine properties to MSCs. In accordance with the present invention, there is provided a method whereby MSCs are vaccinated by, but not limited to, commercially available vaccines including those produced by Pfizer, Moderna, AstraZeneca and Johnson & Johnson either prior to or after IV infusion of MSCs. Also, a further embodiment includes harvesting of MSCs from patients who are or were COVID-19 positive and using the MSCs or derivatives thereof as a COVID-19 treatment. The MSC derivatives include materials secreted into the extracellular fluid including conditioned medium, purified exosomes, mRNA, microRNA, proteins, or other biologically active molecules. MSC and their derivatives may also be genetically modified to enhance production of virus neutralizing antibodies or to provide other functional benefits.

### BRIEF DESCRIPTION OF THE DRAWINGS

A complete understanding of the present invention may be obtained by reference to the accompanying drawings, when considered in conjunction with the subsequent detailed description, in which:

FIG. 1 are graphs of hospital records showing ICU standard of care measures of pulmonary parameters for a coronavirus patient and treated with the inventive AlloRx Stem Cell® therapy;

FIG. 2 are graphs of hospital records showing ICU standard of care measures of pulmonary function for the same coronavirus patient and treated with the inventive AlloRx Stem Cell® therapy;

FIG. 3 are graphs of hospital records showing ICU standard of care measures of blood coagulation for the same coronavirus patient and treated with the inventive AlloRx Stem Cell® therapy;

FIG. 4 are graphs of hospital records showing ICU standard of care measures of renal function for the same coronavirus patient and treated with the inventive AlloRx Stem Cell® therapy;

FIG. 5 is a graph of hospital records showing ICU standard of care measures of liver function for the same coronavirus patient and treated with the inventive AlloRx Stem Cell® therapy; and

FIG. 6 is a graph of hospital records showing ICU standard of care of nutritional assessment for the same coronavirus patient and treated with the inventive AlloRx Stem Cell® therapy.

FIG. 7 shows the effect of stem cell activation by NutraVivo™ a prototype of Stemulife™ on adipose-derived MSCs extracted from treated and untreated patients.

### DETAILED DESCRIPTION OF EMBODIMENTS

Although the following detailed description contains specific details for the purposes of illustration, those of ordinary skill in the art will appreciate that variations and alterations to the following details are within the scope of the invention. Accordingly, the exemplary embodiments of the invention described below are set forth without any loss of generality to, and without imposing limitations upon, the claimed invention.

A preferred embodiment is treatment of viral pneumonia secondary to COVID-19 using IV infusion of mesenchymal stem cells in patients receiving COVID-19 vaccination. Presently, several different COVID-19 vaccines are in development at various stages of regulatory approval. The following products have received US regulatory authorization for human vaccination: Pfizer, Moderna, Johnson & Johnson, and Astra Zenica COVID-19 vaccine. Several other vaccines are in pre-clinical, clinical, or developmental stages. Hence the term COVID-19 vaccine as used herein does not limit the particular vaccine to be used with MSCs. Furthermore, the present invention is not limited to treatment of COVID-19 infections or those caused by mutated variants of COVID-19 but is inclusive of any viral infection causing ARDS including SARS-CoV, SARS-CoV-2 (COVID-19), MERS-CoV, influenza viruses including H5N1, herpes simplex virus, cytomegalovirus (CMV) and other known and unknown viruses that induce ARDS.

Different treatment protocols may be appropriate for the stages of symptom severity in COVID-19. Many asymptomatic patients recover without serious illness. A preferred embodiment for adjuvant therapy to diminish allergic reactions to vaccine including anaphylaxis, reduce inflammation, and provide therapy to COVID-19 variants is concomitant administration of mRNA-based vaccines together with IV infusion of MSCs preferably at 1-2 million MSCs/Kg body weight although other dosages are embodied as well such as 3 to 5 million MSCs per patient given that adjuvant therapy follows the IV infusion.

More severe cases have higher mortality and MSC therapy appears to be effective in these cases. Hence, a preferred patient population is intubated patients at 3 to 10 days or, more preferably, 3 to 5 days on assisted mechanical respiration prior to initial administration of MSCs. The MSCs are typically administered at 100 to 150 million cells by standard methods of intravenous infusion in 50 to 100 ml normal saline that may be repeated within the next 3 to 5 days as determined by clinical assessment of the patient. Also, since there is evidence of recovery from multi-organ failure by treatment of COVID-19 patients with MSCs (Examples 1 to 6), it is likely that MSC therapy may be effective in treating persistent symptoms following recovery from COVID-19 infections, sometimes referred to as “long-COVID” aka PASC (post-acute sequelae SARS-CoV-2 infection). A further embodiment, therefore, is use of MSC IV infusion to treat long COVID. This may also occur through use of MSC derivatives for long COVID-19 therapy including conditioned media collected from cultured MSCs containing unpurified exosomes, purified exosomes, MSC-derived RNA, microRNA including miR133b, miR-15a, miR-15b, miR-16, miR-30, miR-let7 (Asgarpour, et al, Cell Communication and Signaling (2020) 18: 149). MSC derivatives are deployed to patients in need by well-known methods to those skilled in the art including IV infusion and local injections.

While a variety of different tissue-derived MSCs is used clinically, the preferred embodiment is use of human umbilical cord-derived MSCs because of numerous advantages thereof, including regulatory compliant donor screening, commercially advantageous manufacturing, significantly improved potency compared to MSCs derived from other tissues, (Riordan N H, et al, J Transl Med. 2018; 16(1):57). A further preferred embodiment is the use of mesenchymal stem cells in Lot CT121816CT deposited according to ATCC Accession number PTA-124321.

In addition, the current invention embodies therapy of Long COVID (PASC) by umbilical cord-derived MSCs together with adjuvant therapies given before, during or after IV infusion of MSCs. Such adjuvants include agents that activate specific properties of MSC that enhance therapeutic benefit such as enhanced proliferation, migration, potency and gene expression. The assignee has commercialized such a product with active ingredients that include curcumin, quercetin, CoQ10, resveratrol and piperine now marketed as Stemulife™, that has been shown to increase proliferation and migration of MSCs as well as inducing significant increases in MSC expression of SirtI (Sirtuin I, anti-aging gene), CXCR4 (Chemokine receptor 4, that drives MSC migration), Oct3/4 (Octomer binding transcription factor 3/4, a pluripotency gene), Hsp70 (Heat shock protein 70, a cellular protective chaperone), and FGF21, (fibroblast growth factor 21, a member of the fibroblast gene family) (https://www.vitrobiopharma.com/blogs/white-papers/stem-cell-activation-by-natural-products-blog). Stemulife™ at recommended dosage is a specific adjuvant embodied as a combination therapy together with IV infusion of umbilical cord-derived MSCs in the treatment and management of long COVID (PASC). A further embodiment includes adjuvant biological agents enhancing MSC therapeutic effect including but not limited to micro RNA: miR-133b, miR-15a, miR-16, miR-30 (Asgarpour, et al, Cell Communication and Signaling (2020) 18: 149) and other biological agents resulting in enhanced proliferation, migration, potency and increased mitochondrial content or function of UC-MSCs.

A further embodiment is treatment of viral pneumonia with MSCs derived from patients who have been exposed to the COVID-19 virus. Treatment includes use of the MSCs alone or preferably using a conditioned medium derived from nearly confluent patient-specific MSCs collected by methods well-known to those skilled in the art. Furthermore, purified exosomes secreted from the prior COVID-19 positive patient-specific MSCs may be used as the therapeutic agent or other biologically active substances secreted from cultured stem cells such as mRNA, microRNA, and proteins with specific activation of MSC functional properties

The invention is also treatment of viral pneumonia with MSCs that are genetically modified as by CRISPR technology, intracellular delivery of mRNA, micro RNA: miR-133b, miR-15a, miR-16, miR-30, DNA and other methods well-known to those skilled in the art. While any such genetic modification is encompassed herein, preferred or typical alternations result in increased expression of viral specific proteins including, without limitation, the spike protein of COVID-19 or its amino acid derivatives and other COVID-19 antigens, mRNA containing liposomes especially those containing the alternatively spliced Dicer isoform for virus-derived siRNA production, antiviral Dicer (avD) (Poirier, E Z, et al, Science 373, 231 (2021); miRNA activating RNAi 4; the following micro RNAs: miR-133b, miR-15a, miR-16, miR-30 mc133b and epigenetic agents resulting in activation of these signaling molecules, agents enhancing mitochondrial content or function in MSCs. Preferred embodiments of bioengineered MSCs include increased expression of prostaglandin E2 (PGE2), chemokine receptor 4 (CXCR4) and other immunomodulation agents.

Furthermore, cancer specific proteins that elicit immune responses in primary tumor cells are a preferred embodiment including, without limitation, prostate-specific antigen (PSA), carcinoembryonic antigen (CEA), CA-125, and CA19-9.

The following examples are intended to further illustrate the invention and its more typical embodiments. They are not intended to limit the invention in any manner.

### Example 1

The assignee, Vitro Biopharma, a Golden, Colo. based stem cell laboratory, has received FDA authorization for compassionate use of its MSCs (AlloRx Stem Cells®) as an Emergency Investigational New Drug (eIND #22262). The first patient to be treated was admitted to an emergency room with classic COVID-19 symptoms in late April 2020 and had several comorbidities prior to admission including diabetes and heart disease. While intubated in the ICU, the patient's condition worsened using the standard of care and treatment with convalescent plasma. The patient's kidney and liver function began to fail, requiring dialysis. Additionally, the patient experienced sepsis and a stroke while in the ICU and was comatose for almost seven weeks.

Following the treatment with AlloRx Stem Cells® by IV infusion of 100,000 cells at days 8, 12 and 14, the patient experienced resolution of multiple organ failure, recovery from coma, and restoration of neurological, pulmonary, liver, and renal function. The patient was removed from assisted respiration and dialysis. The patient now eats, drinks, and speaks, and has regained motor function. Measures of inflammation (serum CRP levels) that were elevated at admission and rose during ICU treatment returned to normal levels. The patient has recently been discharged from ICU is at home and is currently undergoing physical therapy to recover from the stroke suffered while in the ICU.

Donor screening, manufacturing and quality control occurred by cGMP compliant methods that were reviewed and approved through FDA review of the CMC section of FDA-authorized IND #20503 prior to the eIND authorization (#22262) to treat this patient. This product was also manufactured in full compliance with ISO9001:2015 and ISO13486:2016

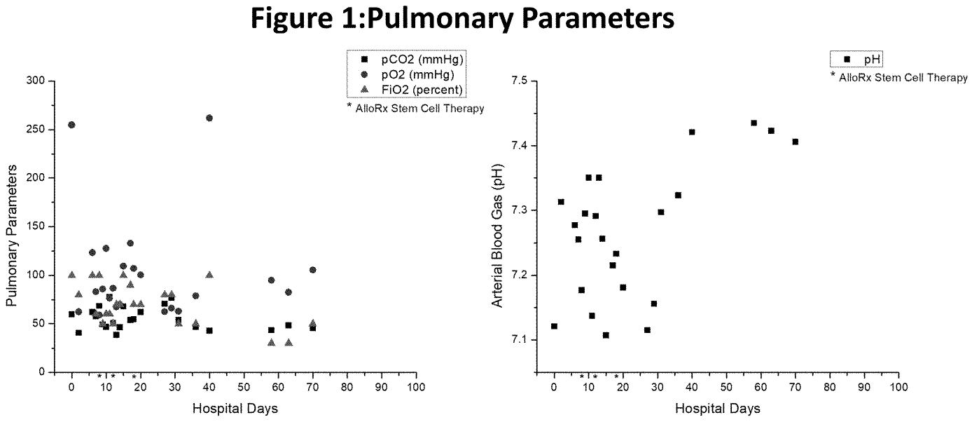

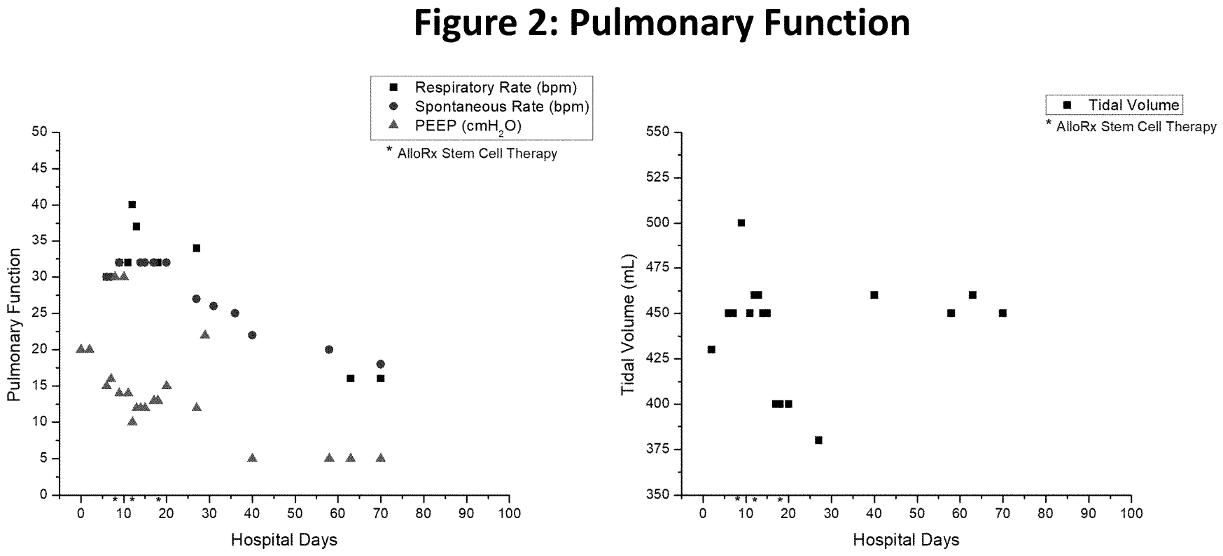

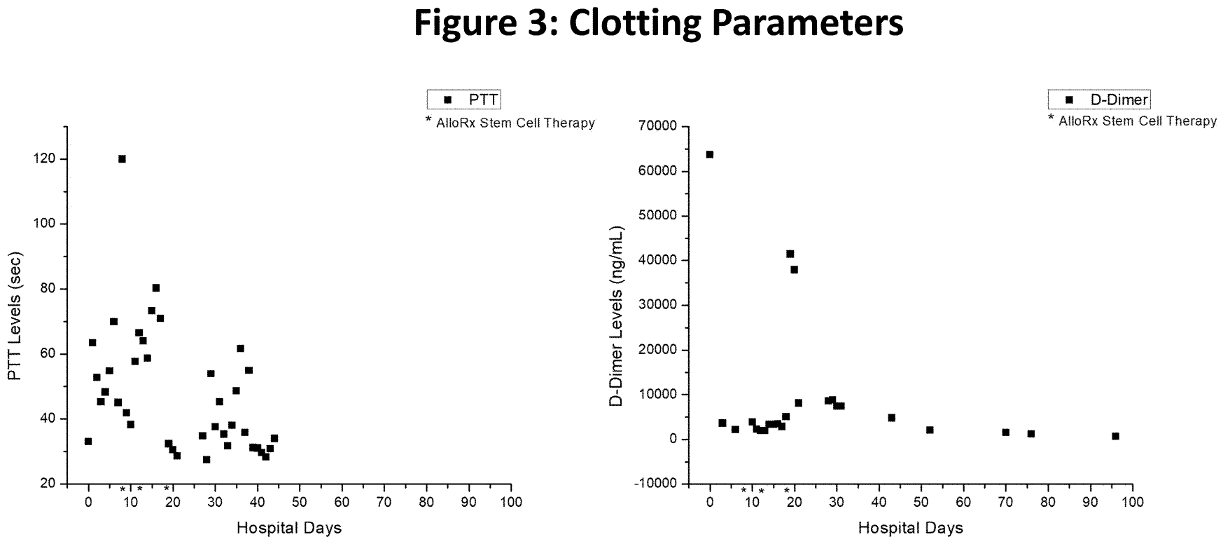

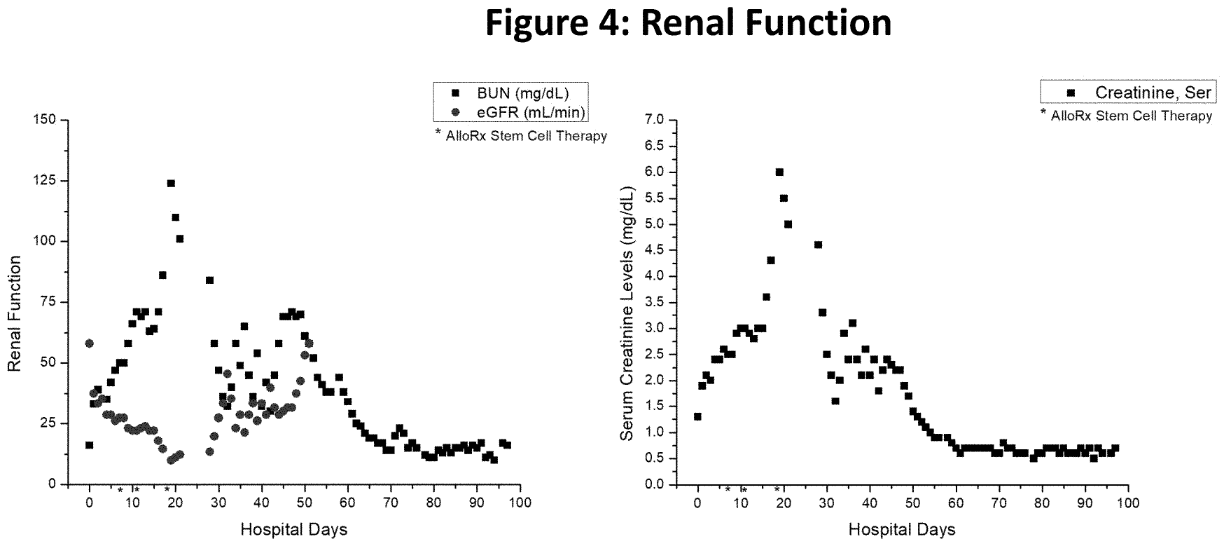

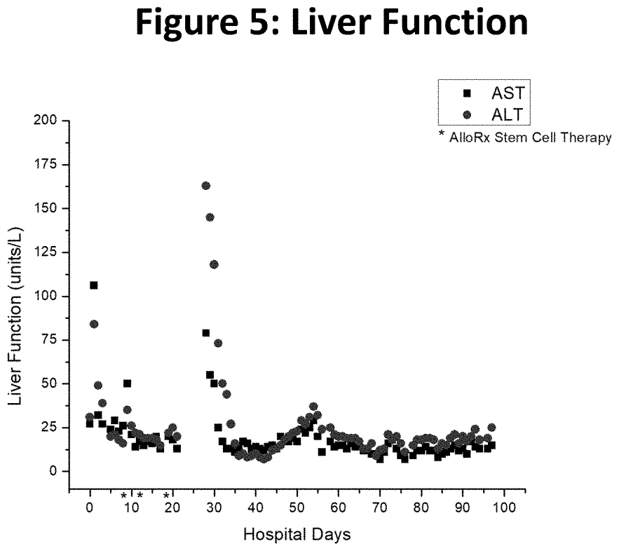

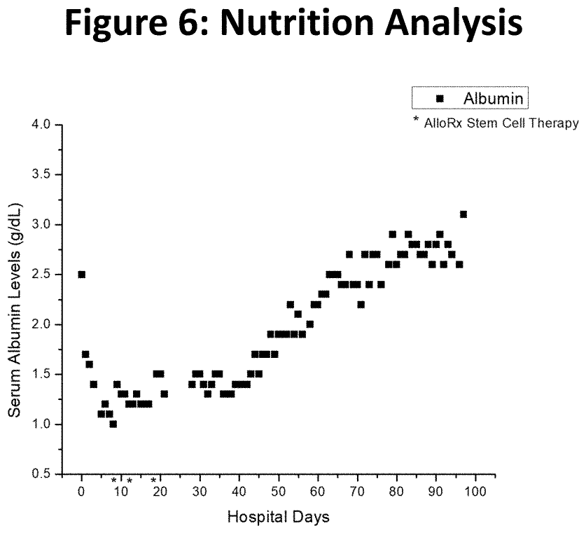

The graphs shown in FIGS. 1-6 show the results of ICU monitoring of the patient's clinical status before and after the infusion of three separate doses of 100 million AlloRx Stem Cells®. The results show the patient's pulmonary, liver, renal, coagulation, and nutritional status monitored over a continuous period of 100 days and resolution of sepsis, renal, liver and pulmonary failure.

To date, AlloRx Stem Cells® have been employed in eight patients with COVID-19 with recovery from COVID-19 symptoms in all patients and recovery from multi-organ failure in one patient. Other than COVID-19 treatments, AlloRx Stem Cells® have been used to treat 200 total patients for various medical conditions without safety concerns and evidence of efficacy.

This example illustrates the utility of the embodiments of the present invention including MSC (AlloRx Stem Cells®) therapy of ARDS secondary to COVID-19 infection and effective therapy of PASC, long COVID as well since multi-organ failure was resolved following IV infusion of AlloRx Stem Cells®

### Prospective Example 2: Immunoassay for UC-MSCs Exposed to SARS-CoV-2 Version 09222021

### 1.0 Purpose

To determine if umbilical cord MSCs exposed to SARS-CoV-2 are able to provide an immune response and provide adaptive immunity through a vaccine like process.

### 2.0 Test Articles

The test articles for this experiment will include umbilical cord mesenchymal stem cells (UC-MSCs) exposed to SARS-CoV-2 virus (Catalog SC00A1-CV), naïve B cells, naïve T cells, and PBMCs from unvaccinated/unexposed donors (three different donors), and adapted B cells, adapted T cells, and PBMCs from vaccinated donors (three different donors).

### 3.0 Procedure

3.1 When ready to perform the experiment, allow water bath to equilibrate to 37° C. Obtain the cells from storage.
3.2 Place the vial into the water bath and provide continuous agitation, e.g., swirling, to the vial while it is submerged in the 37° C. water bath. Continue with agitation until the cells are completely thawed and no ice remains within the cell suspension, usually about 1 to 2 minutes.
3.3 Establish the UC-MSCs initial passage culture at a plating density of about 5,000 to 10,000 cells/cm² in suitable tissue culture dishes, plate or flasks. Add the appropriate volume of MSC culture medium to the plate or flask to be used for culture.
3.4 Co-culture UC-MSCs as follows for 7 days:
   3.4.1 UC-MSCs+Naïve B Cells (Donor A)
   3.4.2 UC-MSCs+Naïve T Cells (Donor A)
   3.4.3 UC-MSCs+Naïve PBMCs (Donor A)
   3.4.4 UC-MSCs+Naïve B Cells (Donor B)
   3.4.5 UC-MSCs+Naïve T Cells (Donor B)
   3.4.6 UC-MSCs+Naïve PBMCs (Donor B)
   3.4.7 UC-MSCs+Naïve B Cells (Donor C)
   3.4.8 UC-MSCs+Naïve T Cells (Donor C)
   3.4.9 UC-MSCs+Naïve PBMCs (Donor C)
   3.4.10 UC-MSCs+Vaccinated B Cells (Donor A)
   3.4.11 UC-MSCs+Vaccinated T Cells (Donor A)
   3.4.12 UC-MSCs+Vaccinated PBMCs (Donor A)
   3.4.13 UC-MSCs+Vaccinated B Cells (Donor B)
   3.4.14 UC-MSCs+Vaccinated T Cells (Donor B)
   3.4.15 UC-MSCs+Vaccinated PBMCs (Donor B)
   3.4.16 UC-MSCs+Vaccinated B Cells (Donor C)
   3.4.17 UC-MSCs+Vaccinated T Cells (Donor C)
   3.4.18 UC-MSCs+Vaccinated PBMCs (Donor C)
3.5 Collect conditioned media of all cultures. Freeze and store for later analysis. (Analysis input for conditioned media to measure antibody secretion RT-PCR)
3.6 Subculture cells according to subculture protocol.
3.7 Immunocytochemistry T cells for SARS-CoV-2 antibody
3.8 Flow Panels pre- and post-co-culture analysis
Gating out stem cells (MSCs at acquisition point)
Multiple panels.2 panels initially. PBMC done separately with specific panels. CD69/CD25 T/B-cell activation, HLA-DR, CD38—B-cell differentiation into plasma blasts, CD3/4/8/19 lineage markers, CD45RO/RA.

| | BD FACSCanto II Detector | | | | | | | |
|---|---|---|---|---|---|---|---|---|
| | Panel: | | | | | | | |
| | 405 B | 405 A | 488 E | 488 D | 488 B | 488 A | 633 C | 633 A |
| — | (BV421) | (BV510) | (FITC) | (PE) | (PerCP) | (PE-Cy7) | (APC) | (APC-H7) |
| — | — | — | — | — | — | — | — | — |
| VITRO-1 | CD69 | HLA-DR | CD19 | CD25 | CD3 | CD8 PE- | CD38 | CD4 APC- |
| | BV421 | BV510 | FITC | PE | PerCP | Cy7 | APC | Cy7 |
| VITRO-2 | CD45RA | HLA-DR | CD19 | CD45RO | CD3 | CD8 PE- | CD38 | CD4 APC- |
| | BV421 | BV510 | FITC | PE | PerCP | Cy7 | APC | Cy7 |

We expect upregulation in T and B cells and PBMCs and HSCs in the naïve population similar to the vaccinated populations thus showing vaccine-like antibody responses in the naïve population to Sars CoV-2.

### Example 3: Activation of Patient MSCs by Use of StemuLife™

Fat was donated by 3 female patients processed for the extraction of AD-MSCs using collagenase extraction. One patient was on NutraVivo™ Dietary Supplements (NVF0525) for 6 months prior to fat collection. NutraVivo™ dietary supplements were an early prototype of StemuLife™. We compared the total cell counts, viability, total and viable mononuclear cells, doubling time, in isolated AD-MSCs post expansion between the NutraVivio™ patient and control patients. All AD-MSCs were analyzed through passage 2 (P2).

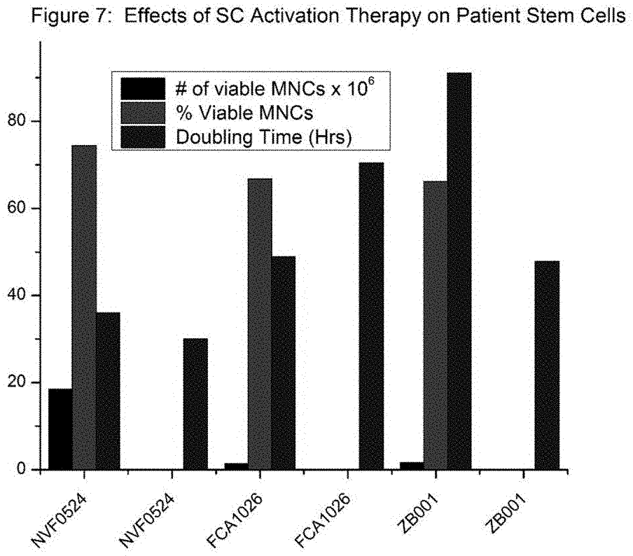

The results shown in FIG. 7 show the effect of NutraVivo™ Stem Cell Activation Therapy on stem cells derived from patients. Patient with ID number NVF0524 was treated with NutraVivo™ Brain Grow Activator, Brain Grow Stimulator & Brain Grow Energizer for 6 months according to recommended dosage while patient ID numbers FCA1026 & ZB001 did not have the NutraVivo™ stem cell activation therapy. The NutraVivo™ stem cell activation therapy increased the number of recovered viable MNCs by more than 10-fold at higher viability and with lower doubling times (blue bars). These results show that NutraVivo™ stem cell activation therapy improves the quality of stem cells derived from fat derived from a patient. These results are further supported by in-vitro studies of AlloRx Stem Cells® showing increased proliferation, migration and statistically significant increased expression of Sirt I (300-fold), CXCR-4 (20-fold), Oct 3/4 (20-fold), Hsp70 (20-fold) ((https://www.vitrobiopharma.com/blogs/white-papers/stem-cell-activation-by-natural-products-blog). The results obtained from the in-vitro, cell-based assays are also supported by the results obtained from a patient treated with Brain Grow Activator, Brain Grow Stimulator and Brain Grow Energizer. The later provides general increases in cellular energy by promoting mitochondrial function. This patient was on the formulation for six months and then had their stem cells processed from fat derived through liposuction. Analysis of these stem cells compared to untreated patients supports the results of cell-based assays. We saw a greater than 10-fold increase in the number of viable MNCs recovered from the lipoaspirate as well as a 2-fold increase in the rate of MSC proliferation (FIG. 7). The former result is likely due to increased stem cell content within fat tissue due to increased stem cell proliferation as demonstrated in the cell-based assays. Also, the increased MSC proliferation rate reflects increased stem cell potency. This patient also received numerous stem cell transplants and exhibited diminished systemic complications of Parkinson's disease including diminished GERDS, improved dyspnea and swallowing reflexes.

Since other modifications and changes varied to fit particular operating requirements and environments will be apparent to those skilled in the art, the invention is not considered limited to the example chosen for purposes of disclosure and covers all changes and modifications which do not constitute departures from the true spirit and scope of this invention.

Having thus described the invention, what is desired to be protected by Letters Patent is presented in the subsequently appended claims. All references, including listed patents and journal articles, are incorporated by reference in their entirety.

What is claimed is:

1. A method of treating a patient having viral pneumonia secondary to COVID-19 comprising administering an IV infusion of mesenchymal stem cells (MSCs) in patients receiving COVID-19 vaccination.

2. The method of treating a patient having viral pneumonia secondary to COVID-19 in accordance with claim 1, wherein the vaccine is selected from at least one vaccine consisting of: Pfizer, Moderna, Johnson & Johnson and Astra Zenica COVID-19 vaccine.

3. The method of treating a patient having viral pneumonia secondary to COVID-19 in accordance with claim 1, wherein the patient is treated with an mRNA-based vaccine followed by IV-infusion of 2 million to 100 million MSCs.

4. The method of treating a patient having viral pneumonia secondary to COVID-19 in accordance with claim 1, wherein the patient requires mechanically assisted breathing.

5. The method of treating a patient having viral pneumonia secondary to COVID-19 in accordance with claim 1, wherein the COVID-19 infection results in symptoms persisting more than one month beyond an initial positive COVID-19 diagnostic test.

6. The method of treating a patient having viral pneumonia secondary to COVID-19 in accordance with claim 1, wherein the mesenchymal stem cells are Lot CT121816CT deposited according to ATCC Accession number PTA-124321.

7. A method of treating a patient having viral pneumonia comprising administering MSCs derived from patients who have been exposed to the COVID-19 virus.

8. The method of treating a patient having viral pneumonia in accordance with claim 7, wherein the MSCs are derived from an umbilical cord of a donor patient who was exposed to the COVID-19 virus.

9. The method of treating a patient having viral pneumonia in accordance with claim 7, wherein a conditioned medium is collected from the MSCs and the conditioned medium is used to treat COVID-19 patients.

10. The method of treating a patient having viral pneumonia in accordance with claim 7, wherein purified exosomes are derived from the MSCs and the purified exosomes are used to treat COVID-19 patients.

11. A method of treating a patient having viral pneumonia comprising administering MSCs genetically modified using CRISPR technology.

12. The method of treating a patient having viral pneumonia with MSCs genetically modified using CRISPR technology in accordance with claim 11, wherein a spike protein of COVID-19 and amino acid sequences thereof are expressed in the MSCs.

13. The method of treating a patient having viral pneumonia with MSCs genetically modified using CRISPR technology in accordance with claim 11, wherein PGE2, CXCR4 or amino acid sequences thereof are expressed in the MSCs.

14. A method of treating a patient with PASC, long COVID-19 by the IV infusion of AlloRx Stem Cells® followed by adjuvant therapy that increases MSC proliferation, migration, potency and epigenetic reprogramming.

15. The method of claim 14, wherein the adjuvant therapy occurs by administration of Stemulife™.

16. The method of claim 14, wherein the adjuvant therapy occurs by administration of conditioned medium derived from umbilical cord MSCs.

17. The method of claim 14, wherein the adjuvant therapy occurs by administration of purified exosomes derived from umbilical cord MSCs.

18. The method of claim 17, wherein the umbilical cord MSCs are AlloRx Stem Cells®.

19. The method of claim 14 wherein the adjuvant therapy consists of miRNA including miR-133b, miR-15a, miR-16, miR-30 mc133b and epigenetic agents resulting in activation of these signaling molecules, agents enhancing mitochondrial content or function in MSCs.
